(19) United States
(12) Patent Application Publication
(10) Pub. No.: US 2026/0309596 A1
(43) Pub. Date: Oct. 8, 2026

(54) Preparation of Platelet Free Mononuclear Cells

(71) Applicant: Russell Biotech, Inc., Doylestown, PA (US)

(72) Inventor: Thomas Russell, Doylestown, PA (US)

(73) Assignee: Russell Biotech, Inc., Doylestown, PA (US)

(21) Appl. No.: 18/880,664

(22) PCT Filed: Jul. 3, 2023
(86) PCT No.: PCT/US23/26834

§ 371 Date: Jan. 2, 2025

Related U.S. Application Data
(60) Provisional application No. 63/359,001, filed on Jul. 7, 2022.

(51) Int. Cl. G01N 1/40

(52) U.S. Cl. CPC G01N 1/4077; G01N 2001/4083

(57) ABSTRACT

Platelets are similar in density to PBMC and thus are contaminants of PBMC isolated by Ficoll density gradient centrifugation the established procedure for preparing PBMC. Platelets are very sticky and often interfere with experiments requiring lymphocytes and or monocytes (PBMC). Methods, compositions and kits are provided for the rapid removal of the undesired platelets from PBMC yielding platelet free PBMC in high yield. The selection process, in the preferred embodiment of the invention, is based on magnetic metallic particles that are uniquely suited for removal of platelets from whole blood or PBMC. The preferred particle is composed of nickel metal in a preferred size range of 0.8-3.5 micron. Specific anti-platelet binding agents are bound to the nickel particles. The particles are mixed with the sample rapidly without non-specifically binding to non-targeted cells. The sample is then placed in a magnetic field and the platelets bound to the magnetic particles are very rapidly removed yielding platelet free whole blood or PBMC in very high yield for further biological experimentation. The method also provides for the removal of platelet and platelet fragments from PBMC preparations where platelets may be present prior to freezing or after thawing the PBMC preparation using a three-bead combination anti-platelet magnetic particle.

### CROSS REFERENCE

This application is the US national application of PCT/2023/026834, filed 3 Jul. 2023, which claims benefit of and priority to U.S. Provisional Patent Application No. 63/350,001, filed 7 Jul. 2022, where permissible incorporated by reference in its entirety.

### BACKGROUND

### 1. Field of the Invention

The invention provides novel means and methods for eliminating platelet contamination from peripheral blood mononuclear cells (PBMC). The invention relates more specifically to kits and reagents to remove platelets from whole blood or PBMC using magnetic separation and/or gravity settling.

### 2. Description of Related Art

PBMC are comprised of desired lymphocytes and monocytes and are used routinely in biological research including immunological research. PBMC are isolated from whole blood using Ficoll gradient centrifugation which removes granulocytes and red blood cells but not platelets yielding the desired lymphocytes and monocytes contaminated with platelets. Numerous references (see other publications: 1-10) provide means (primarily further centrifugation and washing steps) to remove the undesired platelets. Platelets are very sticky and as seen in the cited references often interfere with experiments requiring lymphocytes and or monocytes. Just a few examples are needed to reveal how undesirable platelets are. From references: A Method for the preparation of mononuclear cells devoid of platelet contamination; Artefactual low lymphocyte activity caused by platelet contamination in mononuclear cell preparations; High platelet contamination in progenitor cell concentrates results in significantly lower CD34+ yield after immunoselection; The measurement of transforming growth factor type beta levels produced by peripheral blood mononuclear cells requires the efficient elimination of contaminating platelets and the list goes on.

The only means available to practitioners of the art to remove platelets are different means of centrifugation/washing of PBMC.

The following procedure may also help reduce platelet contamination in mononuclear cell suspensions prepared from fresh whole blood:
1. When isolating the mononuclear cells during density gradient centrifugation (e.g. Lymphoprep™, Serumwerk Bernurg AG or Ficoll™, Cytiva Sweden AB), avoid taking any of the platelet-enriched plasma layer.
2. Next, wash the isolated mononuclear cells:
   Perform a slow spin on the isolated cells (120×g, 10 min, brake off, room temperature)
   Carefully remove the platelet-rich supernatant and discard
   Resuspend the cell pellet in fresh buffer (using a fresh pipette).
   Repeat at least twice for a total of 3 or more washes.

These means are often not very effective in removing platelets, take time and lead to significant loss of PBMC. Thus, there is a definite need for improved methods that yield PBMC devoid of platelets without loss of the much desired PBMC. The inventions(s) described herein solve this problem yielding PBMC in high yield and devoid of platelets.

### SUMMARY OF THE INVENTION

The present invention provides methods and kits to remove platelets from whole blood, diluted whole blood or PBMC that remain part of the PBMC fraction following Ficoll density gradient centrifugation of whole blood. The kits comprise reagents including nano/micro particles bound to antibodies that bind specifically to platelets. Once the anti-platelet particles are bound to the platelets means such as magnetic or gravity are used to remove the platelets from whole blood or PBMC yielding platelet free PBMC in high yield.

A preferred embodiment uses dense metallic nickel magnetic particles as described in U.S. Pat. Nos. 9,435,799 and 9,739,768 incorporated herein by reference. The unique features of the particles (rapid magnetic separation times, rapid mixing times and the ability to work directly in undiluted whole blood with minimal loss of non-targeted cells (here PBMC) solve the problem and consequently yield platelet free PBMC.

Another preferred embodiment uses dense particles as described in U.S. Pat. No. 5,576,185 incorporated herein by reference. Though larger than particles in '799 thus exhibiting slower reaction times the particles can be used to bind to platelets and then by simple gravity settling platelets are removed from whole blood or PBMC.

Another embodiment involves magnetic particles not made of metal but rather composed of metal oxides 50 nm or less in diameter i.e. inorganic compounds (primarily iron oxides) imbedded in non-magnetic material as described in U.S. Pat. No. 5,411,863 (Miltenyi); U.S. Pat. No. 5,466,574 (Liberti); U.S. Pat. No. 4,654,267 (Ugelstad) and 4,707,523 (Chang). Magnetic particles available commercially are by and large made of these inorganic iron oxide magnetic particles and are referred to as superparamagnetic particles (Reference 12).

### BRIEF DESCRIPTIONS OF DRAWINGS

FIG. 1. Properties of metal nickel particles used in a preferred embodiment of the invention.

FIG. 2. Depicts the rapid kinetics seen with nickel particles used in a preferred embodiment of the invention. The sample is undiluted whole blood. The analysis is done on a Beckman Coulter 3-part differential analyzer. The granulocytes are removed using a particle of the invention coupled to CD15.

FIG. 3. Depicts the high recovery of non-targeted cells seen with particles used in a preferred embodiment of the invention as compared to iron oxide superparamagnetic particles of the art. Source: BD Biosciences Catalog. RBI: Russell Biotech, Inc.

FIG. 4. Gravity settling particles of the invention effectively remove platelets from PBMC.

FIG. 5. Properties of inorganic iron oxide particles defined as superparamagnetic particles that are currently available to the market.

### DETAILED DESCRIPTION OF PREFERRED EMBODIMENTS

The means to obtain platelet free PBMC as detailed in this invention comprise three separate methods all based on nano/microparticle technology. The methods include two separate magnetic particles whereby the platelets are removed by a magnetic field and a dense particle whereby the platelets are removed by simple gravity sedimentation or accelerated gravity sedimentation (centrifugation). In all of the methods of the invention the particles have bound thereto anti-platelet antibodies such as CD41 and CD61, but not limited to, that bind to platelets.

The antibodies used in this invention can be either polyclonal or monoclonal. Such monoclonal antibodies (Mab) are commercially available from a number of suppliers. Mab are coupled to the particles of the invention by means known in the art including adsorption and numerous covalent coupling procedures. The Mab can also be coupled to the particle using the biotin/streptavidin/avidin system as known in the art where usually the streptavidin/avidin is bound to the particle and the anti-platelet antibody is coupled to biotin. Other coupling pairs known in the art are included in the invention.

The sample is whole blood (diluted or un-diluted) or PMBC. Whole blood can be human or animal such as mouse or rat but not limited to any mammal having a platelet population in blood.

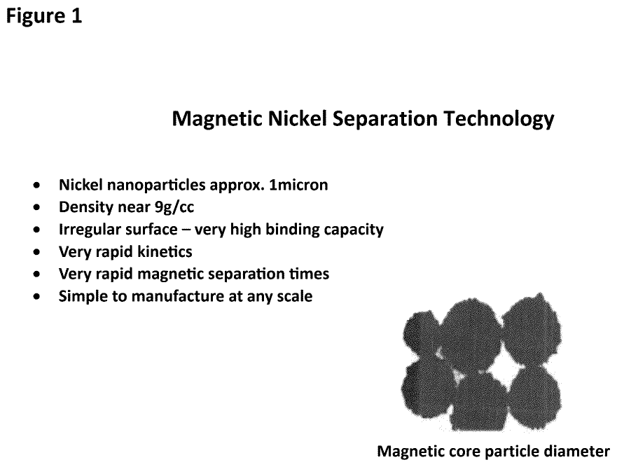

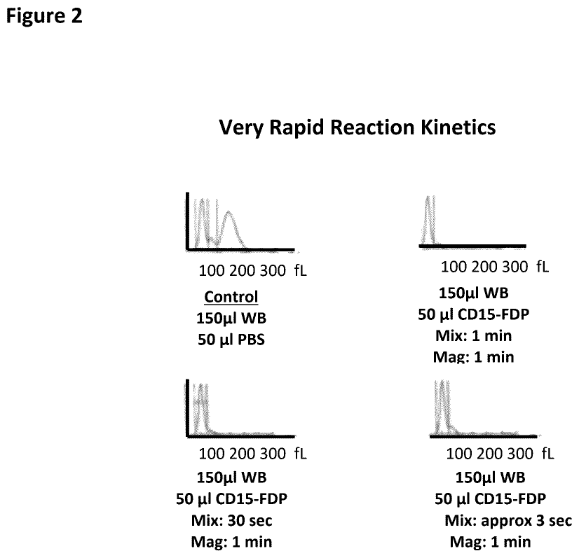

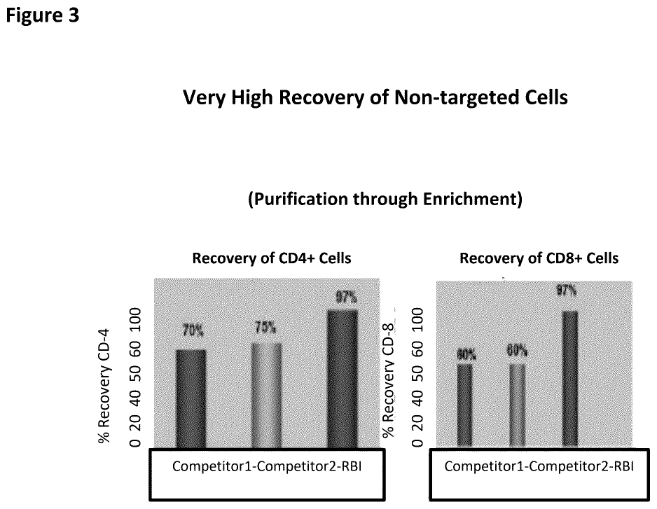

The preferred embodiment uses a dense magnetic metallic particle as described in Russell '799. The particle can range in diameter from 0.3 micron to 3-4 micron with a preferred diameter around 1 micron. The particle is dense having a density of 5-10 g/cc with a preferred density around 9 g/cc. Any metal or metal alloy that is magnetic will function in the invention with a preferred metal being nickel. Any metal commercially available that has the properties identified here such as from Sigma Aldrich but not limited to will function in the assay but the preferred nickel particle is that described in Russell '799. FIG. 1 details features of a preferred nickel particle that make it especially suited to remove platelets including the ability to work easily in undiluted whole blood rapidly with rapid magnetic separation times (FIG. 2). FIG. 2 demonstrates the rapid removal of a cell population (granulocytes) directly from an un-diluted whole blood sample in very rapid mixing times using a magnetic particle as in this preferred embodiment. Another key feature of these particles is the ability to remove a desired cell population with high, almost quantitative recovery, of non-targeted cells (FIG. 3).

In the preferred method, undiluted or diluted whole blood will be mixed directly with the anti-platelet magnetic nickel particles by end-over-end mixing using a device from airbiotech, Inc. for volumes greater than 1 ml and vortexing for volumes equal to or less than 1 ml for the shortest time possible for binding to the platelets in the range of a few seconds to 10 minutes but not limited to. For end-over-end mixing the preferred rotation speed is around 15-30 rpm for 1 micron nickel particles. Following mixing the sample will be placed in a magnetic field depending on the sample volume for a few seconds up to 5 minutes. Suitable magnets are known in the art such as those from Dexter Magnetics. The whole blood sample depleted of platelets will be removed while the sample chamber remains in the magnetic field and then separated on Ficoll to yield the much-desired platelet free PBMC in very high yield for further research studies.

As mentioned here any procedure known in the art for coupling antibody to the particles is incorporated herein i.e. adsorption or covalent coupling. For the preferred embodiment disclosed here adsorption of the anti-platelet antibody directly to the nickel particle surface is simple and results in a very stable particle antibody complex when the isotype of the antibody is an IgM. Anti-CD41 and anti-CD61 IgM isotypes are commercially available i.e. BioLegend. Any antibody isotype i.e. IgM or IgG but not limited thereto that adsorbs to the particle and effectively removes platelets is considered by the disclosure herein. Though experimental studies may be needed to obtain the optimal amount of antibody per particle routinely labelling at 2 mg antibody/meter squared particle surface (surface area) is usually sufficient.

It is best to start with whole blood since any magnetic particles with or without platelets bound thereto that may remain in the sample following placement in the magnetic field (carry over) will be removed following the Ficoll density gradient centrifugation because of the density of the nickel particle relative to cells thus yielding truly platelet free PBMC. However, platelets can be removed, using particles of the invention, directly from PBMC. Any residual particles can then be removed by simple centrifugation at speeds that sediment the very dense particles but do not sediment PBMC. The speeds and times of centrifugation can be easily determined by means known in the art.

A second embodiment uses dense particles such as those described in U.S. Pat. No. 5,576,185 incorporated herein by reference and sold by Novamet. The particles are dense with densities in the range 5-10 g/cc with a preferred density around 9 g/cc. The particles can be any metal, metal alloy or ceramic that can settle by gravity in whole blood or PBMC. The preferred particle diameter is 3-10 micron. Though any diameter particle that operates as described herein is covered by the invention. The method involves dense particles with anti-platelet antibodies bound thereto as described herein (see preferred embodiment). The particles can be added directly to whole blood or whole blood can be added to particles that are at the bottom of the mixing container. The particles are mixed by end-over-end mixing at about 5-10 rpm for volumes greater than 1 ml and by vortexing for volumes of 1 ml or less. Following the mixing step, the sample container is simply placed in an upright position and the dense particles settle by gravity to the bottom of the tube. The gravity settling can be accelerated by centrifugation at speeds that settle the dense particles but do not significantly settle the whole blood. Depending on the sample volume settling times can range from 1-10 minutes. For dense particles that are also magnetic such as nickel particles a magnet can be placed at the bottom of the tube to hold the particles in place while the platelet free blood is removed and then centrifuged over Ficoll to obtain platelet free PBMC. Any residual particles carried over, especially non-magnetic dense particles, will be removed by the centrifugation over Ficoll. Again, it is best to start with whole blood so that any residual dense particles can be removed in the Ficoll centrifugation step. However, the starting material can be PBMC with removal of any residual dense particles due to carryover by a very rapid centrifugation step that leads to settled dense particles without settling of PBMC.

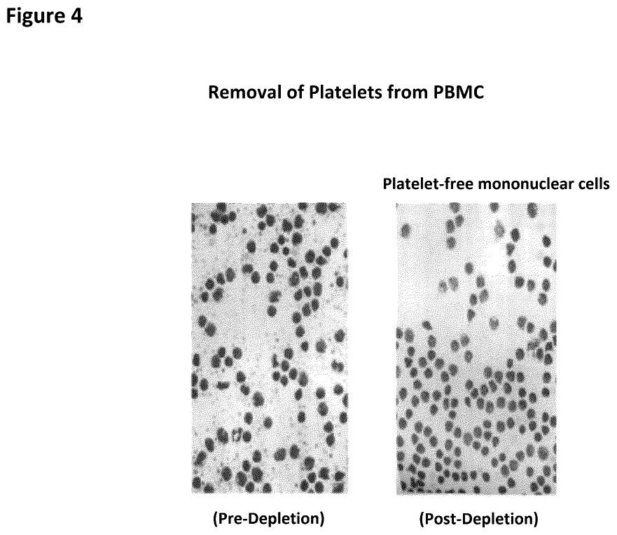

The results of a typical experiment are seen in FIG. 4 which shows the result of treating PBMC with 3.5 micron nickel particles with anti-platelet antibodies bound thereto using the method of gravity settling as described herein. The platelets (small objects seen in the left figure) have been removed (right figure) following the gravity settling procedure.

Prior to adding anti-platelet antibodies to nickel particles the nickel particles are weighed on a scale preferably in a hood while wearing a face mask. Though nickel has GRASS status, while working with nickel powder, one should avoid breathing dust from the particles. Weigh out the amount of nickel desired based on coating antibody at 2 mg/meter squared surface area of the particle. The nickel particles are then heated at 250 degrees centigrade for 3 hours up to 3 days. This accomplishes the following important features: the particles are sterile if needed and an oxide coating covers the outer layer of the particle which significantly reduces the release of nickel ions from the particle and the nickel oxide surface can be used to covalently bind antibodies to the particles by means known in the art. Once the particles are in a buffer solution no more dust is present. A hood and/or mask are no longer required.

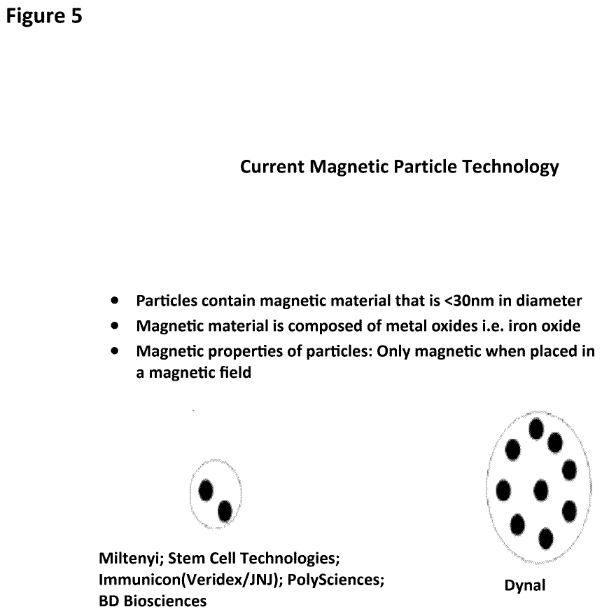

Another embodiment involves use of magnetic particles currently on the market supplied by numerous vendors including Miltenyi Biotec, BD Biosciences, TheromoFisher, EMD Millipore, PolySciences and Stem Cell Technologies to remove platelets from whole blood or PBMC. These particles differ from the magnetic metal particles of '799. They are superparamagnetic particles that are made up of inorganic metal oxides rather than metal. The oxide is usually iron oxide but any metal oxide that is magnetic falls under this embodiment. The metal oxide is para/superparamagnetic because the metal oxide crystal is under 50 nm in diameter (see U.S. Pat. Nos. 5,466,574; 5,411,863; 4,654,267). The metal oxide crystals are embedded in polymeric material by means known in the art to build particles of various diameters from 100 nanometers to 4.5 micron (see FIG. 5). The smaller the particle diameter the more rapid the binding kinetics but as the particle decreases in diameter they are weaker magnetically often requiring complex means for magnetic separation (induced magnetic field and columns are often required). The density of these particles is less than 2 g/cc and thus mixing in undiluted whole blood is problematic and it is usually recommended that prior to adding magnetic particles to whole blood the blood is diluted and or red cells are lysed (Miltenyi Biotec).

Though the providers of superparamagnetic particles offer numerous particles with antibodies bound thereto such as CD15, CD14, CD4, CD8, etc., CD41 and CD61 (anti-platelet) are noticeably absent with the exception of Miltenyi Biotec that offers CD41 and CD61 particles for removal of megakaryocytes and platelets from PBMC preparations. Thus, it appears that the use of superparamagnetic particles of the art for the removal of contaminating platelets from whole blood, as described in this invention, is unobvious as these particles have been available to the market for over 35 years. Most likely this is due to the fact that the particles do not work well in undiluted whole blood. However, the particles do work in diluted whole blood and in PBMC.

One skilled in the art could easily modify procedures to use superparamagnetic particles for the removal of contaminating platelets to create platelet free PBMC. To the extent that such procedures are created they are definitely anticipated by the invention disclosed herein.

Kits will be provided to the market for using the technology described in this invention to solve a major problem faced by researchers that of obtaining platelet free PBMC preparations. The kits will include both consumable reagents and equipment. The consumables will be particles (magnetic or dense magnetic or non-magnetic) of the invention in a suitable buffer such as Phosphate Buffered Saline (PBS) with protein such as 0.1% BSA, but not limited thereto. It is anticipated the particles will be bottled as 2 ml and 5 ml solutions but not limited thereto. The kit will also contain a suitable rinsing buffer known in the art such as PBS with 0.1% BSA. The equipment will be for a mixing means routinely purchased through Airbiotech or similar vendors and for magnetic particles a magnetic separation means though vendors such as Dextermag.

### A Further Embodiment of the Present Invention Improves Upon the Described Methods and Includes a Frozen Fluid Sample of Whole Blood or any Biological Sample Containing PBMCs and Not Whole Blood or PBMCs That Had Not Been Frozen

It is well known in the art that preferred samples of whole blood and or PBMC can be used fresh and in the case of whole blood, manufacturers recommend that whole blood be used within 8 hours of blood draw but no longer than 24 hours. However, such conditions are often not possible so investigators will routinely Ficoll the whole blood within 24 hours to obtain PBMCs that are then frozen and then thawed at a later date for studies. The Ficolled PBMCs are frozen without removal of platelets. Well known procedures exist in the art for both freezing and thawing Ficoll (density gradient) isolated PBMCs (13). It is also well known in the art that upon thawing cell viability is decreased by as much as 50%. Routinely the dead cells are removed by means known in the art including but not limited to centrifugation/washing.

The reason for the low viability following thawing of PBMCs may well be do the presence of varying number of platelets in the sample prior to freezing and or thawing. Since platelets are similar in density to PBMCs (14) they are enriched following density gradient centrifugation on FICOLL or other gradient material (14) used to isolate PBMCs.

Therefore, a need exists for platelets to be removed before freezing and thawing PBMCs. As disclosed herein platelets are sticky and it makes sense to remove them. Reference 14 details a reason to remove them. The reference details the effect of platelets on mitochondrial (mt) DNA content of PBMCs. The copy number of mtDNA ranges from several tens of thousands to several hundreds of thousands depending on the tissue. The copy number largely determines the cellular ATP production. Therefore, a decrease in mtDNA causes the cellular dysfunction leading to severe symptoms such as mtDNA depletion syndrome (14). Platelet contamination can lead to overestimation of mtDNA content suggesting that platelets be removed before mtDNA determination. Routinely platelets are removed by multiple washings that leads to PBMC loses.

For samples where platelets are not removed before thawing the present invention describes procedures to remove platelets after thawing of PBMC samples. Methods described herein leads to the effective removal of platelets from thawed PBMCs that were previously frozen before removal of platelets. Following thawing of PBMCs by any means known in the art, the thawed PBMCs are processed by the disclosure described herein for fresh PBMCs to yield platelet free thawed PBMCs. As discussed herein platelets can be removed by gravity settling and magnetic separation. It is to be noted that particles of the art based on superparamagnetic particles though in operation are inferior to the particles of the invention.

Accordingly, platelet thawing may lead to breaking up of platelet particles into platelet fragments. These possible platelet fragments, if present, still need to be removed. Their removal will not quantitate the presence of platelet fragments but ensures an enrichment method devoid of a certain amount of platelet fragments. To deplete platelets that have not been frozen the above disclosure using anti-CD41 nickel particles is sufficient. However, to ensure the removal of possible platelet fragments the present invention provides improved methodology.

Thawed PBMCs containing platelets and possible platelet fragments are treated with a three-bead combination to ensure a better chance of removing platelet fragments. As described in the original disclosure nickel magnetic or nickel gravity settling beads are made with not only CD41 but also with CD61 and CD36. Using this bead combination the odds of binding platelet fragments increases as a fragment may lack CD41 but then may have CD61 and/or CD36 present.

While the present invention has been described in terms of its preferred embodiments, it is to be appreciated that the invention is not limited thereby, and that one skilled in the art can conceive of numerous variations and modifications of the invention described herein, without departing from the spirit and scope of following claims.

### CITED REFERENCES

Patents/Patent Applications:

| | | |
|---|---|---|
| 5,576,185 | Coulter | Nov. 19, 1996 |
| 9,435,799 | Russell | Sep. 6, 2016 |
| 5,466,574 | Liberti | Nov. 4, 1995 |
| 5,411,863 | Miltenyi | May 2, 1995 |
| 4,654,267 | Ugelstad | Mar. 31, 1987 |
| 4,707,523 | Chang | Nov. 17, 1987 |
| 9,739,768 | Russell | Mar. 9, 2017 |

### OTHER PUBLICATIONS

1. Lad, P. M. etal; A Method for the preparation of mononuclear cells devoid of platelet contamination; J. Immunological Methods vol 110, p 193 (1988).
2. Pawlowski, N. A etal.; Arachadonic acid metabolism by human monocytes. Studies with platelet-depleted cultures. Journal of Experimental Medicine vol 158, p 393 (1983).
3. Rueda, F, etal; Artefactual low lymphocyte activity caused by platelet contamination in mononuclear cell preparations. American Journal of Hematology vol 31, p126 (1989).
4. Cheng, H. H. etal; Plasma processing conditions substantially influence circulating microRNA biomarker levels https://doi.org/10.1371/journal.pone.0064795 (2013).
5. Goday, A etal; Importance of platelet-free preparations for evaluating lymphocyte nucleotide levels in inherited or acquired immunodeficiency syndromes. Clinical Science vol. 65 p 635 (1983).
6. Reiser, M. etal.; High platelet contamination in progenitor cell concentrates results in significantly lower CD34+ yield after immunoselection Transfusion vol 40 p 178 (2000).
7. Bercovitz, R. S. etal; The pro-inflammatory effects of platelet contamination in plasma and mitigation strategies for avoidance Vox Sanguinis vol 102 p 345 (2012).
8. Urata, M. etal; Platelet contamination causes large variation as well as overstimulation of mitochondrial DNA content of peripheral blood mononuclear cells Annals of Clinical Biochemistry: International Journal of Laboratory Medicine http://journals.sagepub.com/doi/full/10.1258/acb.2008.008008 (2008).
9. Merino, J. et al; The measurement of transforming growth factor type beta levels produced by peripheral blood mononuclear cells requires the efficient elimination of contaminating platelets Journal of Immunological Methods vol 153 p 151 (1992).
10. Casale, T. B. and Kaliner, M. A rapid method for isolation of human mononuclear cell free of significant platelet contamination Journal of Immunological Methods vol 55 p 347 (1982).
11. Cell Separation Methods and Applications; edited by Recktenwald and Radbruch; Publisher: Marcel Dekker, Inc. 1998.
12. Examples of Product Catalogs: Miltenyi Biotec; B D Biosciences; EMD Millipore; Stem Cell technologies; Beckman Coulter, PolySciences.
13. Ramachan, H etal, Optimal Thawing of Cryopreserved PBMCs for Use In High-Throughput Human Immune Monitoring Studies; Cells, vol 1 (3); page 313-324; 2012.
14. Urata, M, et el., Platelet Contamination causses large variation as well as overstimulation of mitochondrial DNA content of PBMCs; Annals Clinical Biochemistry; v45; page 513-514. 2008

What is claimed is:

1. A method for obtaining platelet-free peripheral blood mononuclear cells from a whole blood sample comprising:
a. obtaining a whole blood sample treated with Ficoll to remove granulocytes and red blood cells;
b. mixing a volume with anti-platelet magnetic particles having at least one bead for a period of time to allow the anti-platelet particles to bind to platelets and platelet fragments in a mixture;
c. placing the mixture in a magnetic field for a time sufficient to remove the anti-platelet particles; and
d. separating the platelets and platelet fragments in the mixture wherein a remaining fluid sample contains platelet-free peripheral blood mononuclear cells.

2. The method of claim 1 wherein the treated whole blood sample contains a frozen fluid sample having platelets and PBMCs which when thawed contain platelets and platelet fragments.

3. The method of claim 1 wherein the anti-platelet magnetic particles are dense metallic nickel magnetic particles.

4. An anti-platelet magnetic metallic particle comprising:
a. a nickel particle; and
b. an anti-platelet antibody wherein the antibody is adsorbed directly to the nickel particle surface.

5. The particle of claim 1 wherein the three bead combination includes anti-platelet antibody having Anti-CD41 or Anti-CD61.

6. The particle of claim 1 wherein the three bead combination includes anti-platelet antibody having Anti-CD61 and Anti-CD36.

7. The particle of claim 1 wherein the three bead combination includes Anti-CD41, Anti-CD61 and Anti-CD36.

8. A method for obtaining a platelet-free peripheral blood mononuclear cells from a whole blood sample comprising:
a. obtaining a whole blood sample treated with Ficoll to remove granulocytes and red blood cells;
b. mixing a volume with anti-platelet gravity settling particles having at least one bead for a period of time to allow the anti-platelet particles to bind to platelets and platelet fragments in a mixture;
c. allowing the anti-platelet gravity particles in the mixture to settle for a time sufficient to remove the anti-platelet particles; and
d. separating the platelets and platelet fragments in the fluid sample wherein the remaining fluid sample containing peripheral blood mononuclear cells is platelet-free.

9. The method of claim 8 wherein the treated whole blood sample contains a frozen fluid sample having platelets and PBMCs which when thawed contain platelets and platelet fragments.

10. The method of claim 8 wherein the anti-platelet gravity settling particles are dense metallic nickel magnetic or non-magnetic particles.

11. An anti-platelet magnetic or non-magnetic metallic particle comprising:
a. a nickel particle; and
b. an anti-platelet antibody wherein the antibody is adsorbed directly to the nickel particle surface.

12. The particle of claim 11 wherein the three bead combination includes anti-platelet antibody having Anti-CD41 or Anti-CD6.

13. The particle of claim 11 wherein the three bead combination includes anti-platelet antibody having Anti-CD61 and Anti-CD36.

14. The particle of claim 11 wherein the three bead combination includes Anti-CD41, Anti-CD61 and Anti-CD36.

15. A kit for obtaining platelet-free peripheral blood mononuclear cells in a fluid sample comprising:
a. an anti-platelet magnetic particle having at least one bead; and
b. a Ficoll density gradient.
